(19) United States
(12) Patent Application Publication
(10) Pub. No.: US 2005/0075505 A1
(43) Pub. Date: Apr. 7, 2005

(54) Novel process for the preparation of substantially pure 5-(3,5-dimethylphenoxy)methyl-2-oxazolidinone

(76) Inventors: Biren Jaiprakash Gandhi, Baroda (IN); Samir Rameschandra Shah, Baroda (IN); Trinadha Rao Chitturi, Baroda (IN); Rajamannar Thennati, Baroda (IN)

Correspondence Address: Mark Pohl;Pharmaceutical Patent Attorneys, 4th Floor, 55 Madison Avenue, Morristown, NJ, 07960-7397, US

(21) Appl. No.: 10/501,588

(22) PCT Filed: Jan. 13, 2003
(86) PCT No.: PCT/IN03/00009

(30) Foreign Application Priority Data
Jan. 14, 2002 (IN) 27/MUM/2002

(51) Int. Cl. C07D 263/04

(52) U.S. Cl. 548/229

(57) ABSTRACT

Substantially pure 5-(3,5-dimethylphenoxy)methyl-2-oxazolidinone, a compound of formula (1), is prepared by a novel route, which comprises reacting 3-(3,5-dimethylphenoxy)-2-hydroxypropylamine, a compound of formula (2), or its acid addition salt with a compound of formula (3) (YCOZ) wherein Y and Z are selected from X, CC13CO, 1-imidazolyl or substituted imidazolyl, and OR; wherein X is a halide, preferably chloride, and R is selected from substituted or unsubstituted linear, branched or cyclic alkyl and aryl or heteroaryl radicals. The compound of formula (2) is prepared by treating 2-[(3,5-Dimethylphenoxy)methyl]oxirane with ammonia to yield compound of formula (2), and optionally purifying compound of formula (2) by converting to its acid addition salt.

### FIELD OF THE INVENTION

The present invention relates to a novel process for preparing substantially pure 5-(3,5-dimethylphenoxy)methyl-2-oxazolidinone, a compound of formula 1. 5-(3,5-dimethylphenoxy)methyl-2-oxazolidinone, commonly known as metaxalone (INN Name), a compound of formula 1 is indicated as an adjunct to rest, physical therapy and other measures for the relief of discomforts associated with acute, painful musculoskeletal conditions.

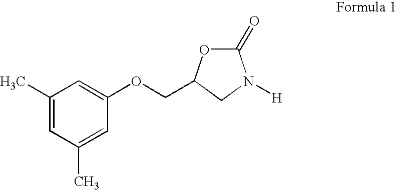

### BACKGROUND OF THE INVENTION

U.S. Pat. No. 3,062,827 generically claims 5-(3,5-dimethylphenoxy)methyl-2-oxazolidinone. This patent also discloses three methods for the preparation of 5-(3,5-dimethylphenoxy)methyl-2-oxazolidinone, viz.
(a) reacting 3-(3,5-dimethylphenoxy)-1,2-propanediol with urea; or
(b) reacting 3-(3,5-dimethylphenoxy)-1-chloro-2-propanol with urea; or
(c) reacting 3-(3,5-dimethylphenoxy)-2-hydroxy-1-propyl-carbamate with urea.

The patent exemplifies the process at elevated temperature i.e. 195-200° C. and also involves distillation of 5-(3,5-dimethylphenoxy)methyl-2-oxazolidinone under high vacuum and temperature. This patent does not disclose the purity of the prepared 5-(3,5-dimethylphenoxy)methyl-2-oxazolidinone; the process described is energy consuming and yields 79% product. When we carried out the patented process the purity of the crude product obtained was only about 51% and unreacted 3-(3,5-dimethylphenoxy)-1,2-propanediol was found to be the major impurity. There is thus a need for a process wherein the starting material is efficiently converted to 5-(3,5-dimethylphenoxy)methyl-2-oxazolidinone.

U.S. Pat. No. 3,446,814 claims a method of preparing 5-(3,5-dimethylphenoxy)methyl-2-oxazolidinone by reacting triglycidyl isocyanurate with m-xylenol The patent exemplifies reacting the raw materials with pulverized sodium hydroxide in chlorobenzene at its reflux temperature which is 131-132° C. for 13 hours in presence of benzyltrimethylammonium chloride, followed by recrystallization of the product from chlorobenzene. This patent does not disclose the purity of 5-(3,5-dimethylphenoxy)methyl-2-oxazolidinone. The process is also energy consuming and yields 76% product.

A novel process has been found for the preparation of 5-(3,5-dimethylphenoxy)methyl-2-oxazolidinone from 3-(3,5-dimethylphenoxy)-2-hydroxypropylamine. None of the methods disclosed in prior art prepare 5-(3,5-dimethylphenoxy)methyl-2-oxazolidinone by the process of the present invention. The novel process converts the starting material to the intended product in an efficient manner such that substantially all of the starting material is converted to the intended product.

### OBJECT OF THE INVENTION

The object of the present invention is to provide a novel process to prepare 5-(3,5-dimethylphenoxy)methyl-2-oxazolidinone.

The process of the present invention provides a novel process that prepares 5-(3,5-dimethylphenoxy)methyl-2-oxazolidinone in high yields in a substantially pure form.

Another object of the present invention is to provide substantially pure 3-(3,5-dimethylphenoxy)-2-hydroxypropylamine, compound of formula 2, or its acid addition salt and the process of its preparation.

### SUMMARY OF INVENTION

The present invention provides a novel process for the preparation of 5-(3,5-dimethylphenoxy)methyl-2-oxazolidinone, compound of formula 1, comprising 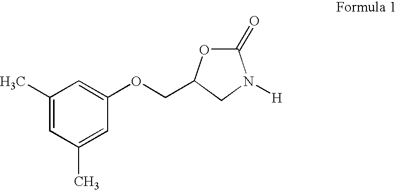
reacting 3-(3,5-dimethylphenoxy)-2-hydroxypropylamine, compound of formula 2, or its acid addition salt with a compound of formula 3, 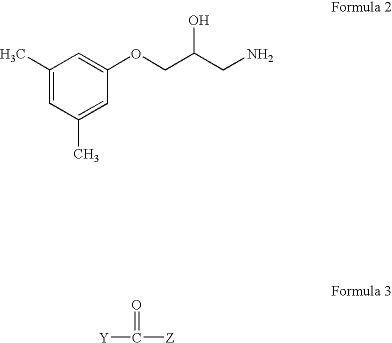
wherein Y and Z are selected from X, CCl₃CO, 1-imidazolyl or substituted imidazolyl and OR; wherein X is a halo radical, and R is selected from a substituted or unsubstituted linear, branched or cyclic alkyl radical, and aryl or heteroaryl radical.

The process of the present invention also provides for the purification of 5-(3,5-dimethylphenoxy)methyl-2-oxazolidinone, compound of formula 1, by crystallization of 5-(3,5-dimethylphenoxy)methyl-2-oxazolidinone (formula 1) from an organic solvent system.

Particularly, the process of the preparation of compound of formula 1 comprises treating compound of formula 5 with a source of ammonia to yield compound of formula 2, optionally purifying compound of formula 2 by converting to its acid addition salt; and 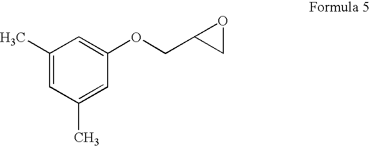
reacting 3-(3,5-dimethylphenoxy)-2-hydroxypropylamine, compound of formula 2, or its acid addition salt with a compound of formula 3.

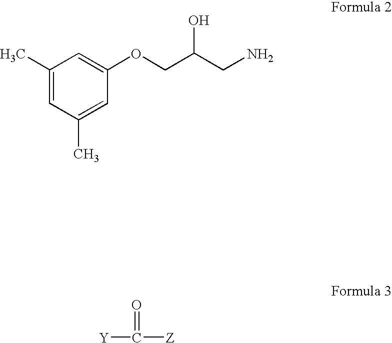

More particularly the process of the present invention for the preparation of 5-(3,5-dimethylphenoxy)methyl-2-oxazolidinone encompasses
(a) reacting 3,5-dimethylphenol, compound of formula 4, with epichlorohydrin and a base to obtain an oxirane, compound of formula 5;
(b) treating compound of formula 5 with a source of ammonia to yield compound of formula 2, optionally purifying compound of formula 2 by converting to its acid addition salt; and 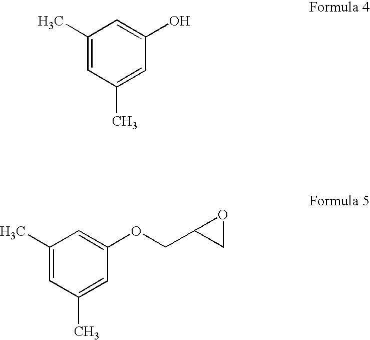
(c) reacting 3-(3,5-dimethylphenoxy)-2-hydroxypropylamine, compound of formula 2, or its acid addition salt with a compound of formula 3.

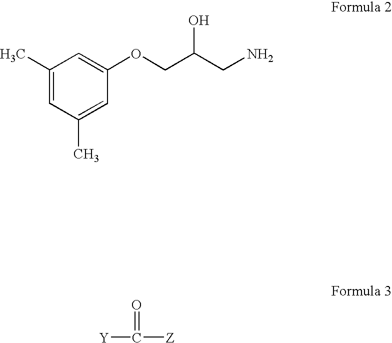

The present invention also provides a substantially pure 3-(3,5-dimethylphenoxy)-2-hydroxypropylamine, compound of formula 2, or its acid addition salt and the process of its preparation comprising treating compound of formula 5 with a source of ammonia to yield compound of formula 2, optionally purifying compound of formula 2 by converting to its acid addition salt.

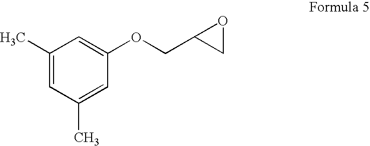

The novel process of the present invention has been found to be advantageous in that the reactions involved can be carried out without substantial expenditure of energy, and the desired product, viz. 5-(3,5-dimethylphenoxy)methyl-2-oxazolidinone (formula 1) can be obtained in high yields in a substantially pure form.

As referred to herein substantially pure 5-(3,5-dimethylphenoxy)methyl-2-oxazolidinone is 5-(3,5-dimethylphenoxy)methyl-2-oxazolidinone with purity greater than 99%.

Preferably the substantially pure 5-(3,5-dimethylphenoxy)methyl-2-oxazolidinone has purity greater than 99.5%, more preferably greater than 99.9% by HPLC.

Most preferably, substantially pure 5-(3,5-dimethylphenoxy)methyl-2-oxazolidinone (formula 1) has a purity greater than 99.5% and has no individual impurity that is more than 0.05% by HPLC.

As referred to herein substantially pure 3-(3,5-dimethylphenoxy)-2-hydroxypropylamine, compound of formula 2, is 3-(3,5-dimethylphenoxy)-2-hydroxypropylamine with purity greater than 99% by HPLC.

### DETAILED DESCRIPTION OF THE INVENTION

A novel method of preparation was conceived and developed by us so as to obtain substantially pure 5-(3,5-dimethylphenoxy)methyl-2-oxazolidinone (formula 1).

The process of the present invention adopts a novel methodology to prepare 5-(3,5-dimethylphenoxy)methyl-2-oxazolidinone which comprises reacting, 3-(3,5-dimethylphenoxy)-2-hydroxypropylamine, a compound of formula 2, or its acid addition salt with compound of formula 3, 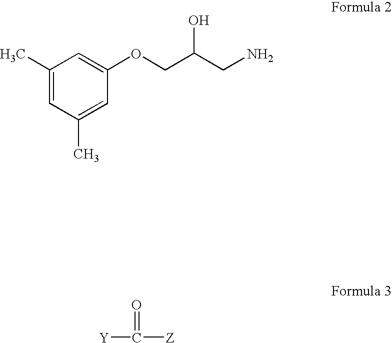
wherein Y and Z are selected from X, CCl₃CO, 1-imidazolyl or substituted imidazolyl, and OR; wherein X is a halo radical, and R is selected from a substituted or unsubstituted linear, branched or cyclic alkyl radical, and aryl or heteroaryl radical.

The substitutions on linear, branched or cyclic alkyl radical comprise of cyano, nitro, alkoxy, aryloxy, mercaptoalkyl, mercaptoaryl, alkyl or arylsulphonyl. The preferred substitutions are the electron withdrawing substituents like cyano or nitro.

The substitutions on aryl, heteroaryl or imidazolyl radical comprise of halo, cyano, nitro, alkoxy, aryloxy, mercaptoalkyl, mercaptoaryl, alkyl or arylsulphonyl. The preferred substitutions are the electron withdrawing substituents like halo, cyano or nitro.

In preferred embodiments, the compound of formula 3 is preferably a carbonate or a haloformate, most preferably a chloroformate.

The present invention also discloses a method for the preparation of 3-(3,5-dimethylphenoxy)-2-hydroxypropylamine, compound of formula 2, which comprises treating compound of formula 5 with a source of ammonia to yield compound of formula 2, and optionally converting compound of formula 2 to its acid addition salt in order to isolate substantially pure compound of formula 2.

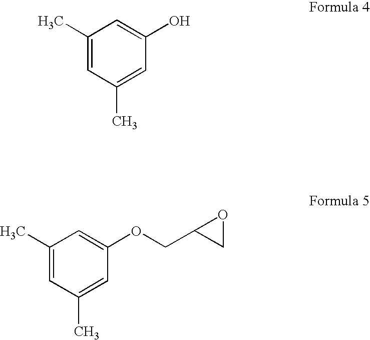

The acid addition salt of compound of formula 2 may be selected from its hydrochloride, sulfate or hydrobromide salt, preferably its hydrochloride salt, in order to isolate substantially pure form compound of formula 2.

As referred to herein substantially pure 3-(3,5-dimethylphenoxy)-2-hydroxypropylamine, compound of formula 2, is 3-(3,5-dimethylphenoxy)-2-hydroxypropylamine with purity greater than 99%.

Details of each step are as given below:

### Preparation of 5-(3,5-dimethylphenoxy)methyl-2-oxazolidinone

According to the process of the present invention, preparation of 5-(3,5-dimethylphenoxy)methyl-2-oxazolidinone is carried out by reacting 3-(3,5-dimethylphenoxy)-2-hydroxypropylamine, compound of formula 2, or its acid addition salt in an organic solvent in the presence of a base, with a compound of formula 3, 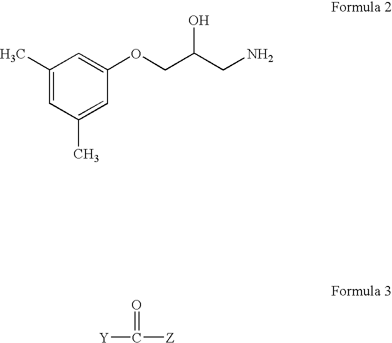
wherein Y and Z are selected from X, CCl₃CO, 1-imidazolyl or substituted imidazolyl, and OR; wherein X is a halo radical, and R is selected from a substituted or unsubstituted linear, branched or cyclic alkyl radical, and aryl or heteroaryl radical.

In preferred embodiments of the invention the compound of formula 3 is a carbonate or a haloformate wherein Y is halo and Z is OR wherein R is selected from linear C₁ to C₄ alkyl radical, preferably a chloroformate, most preferably ethyl chloroformate.

The organic solvent is selected from polar and non-polar solvents comprising of aliphatic, cyclic or aromatic substituted or unsubstituted hydrocarbons such as benzene, toluene, xylene, cyclohexane, dichloromethane, dichloroethane, monochlorobenzene and the like; ketones such as acetone, methlyisobutylketone, methylethylketone, cyclohexanone and the like; cyclic and acyclic ethers such as ether, tetrahydrofuran, dioxan, dimethoxyethane, diglyme and the like; polyethers such as poly(alkylene glycol)s and the like; nitriles such as acetonitrile, benzonitrile and the like; amides such as dimethylformamide, dimethylacetamide and the like. The preferred solvent is an aliphatic, cyclic or aromatic substituted or unsubstituted hydrocarbon, most preferably toluene.

The base for the reaction is selected from a group of organic or inorganic bases. The organic base may be selected from tertiary amines or aromatic bases, and the inorganic base may be selected from bicarbonates, carbonates, hydrides, hydroxides and oxides of alkali or alkaline earth metals. In preferred embodiments the base is an inorganic base, which is a carbonate of an alkali metal, the most preferred base being potassium carbonate.

In the process of the present invention, when the reaction is carried out using an inorgainc base, addition of a facilitator has been found to be very advantageous. The facilitator is a substance that has the property to complex or solvate metal cations, for example, a polyether. Alternatively, the facilitator may be a substance that can
exchange the metal cations with hydrophobic cations, for example, a quaternary ammonium salt or a quaternary ammonium hydroxide where substituents on the nitrogen are selected from alkyl or aralkyl groups, for example, benzyltrialkylammonium halide; or
act in a fashion similar to phase transfer catalyst.

The facilitator may be selected from cyclic and acyclic polyethers. Cyclic ethers such as crown ethers and acyclic ethers such as poly(alkylene glycol) may be used. Poly(alkylene glycol) which may be used is poly(ethylene glycol) (PEG) with an average molecular weight in the range between 200 to 10,000, the most preferred facilitator for the reaction being PEG400.

The reaction can be performed at temperatures ranging from 0 to 150° C. for about 1 to 10 hours, preferably at 50 to 150° C. for about 2 to 8 hours, the most preferred being about 100 to 110° C. for about 5 hours.

For instance, the reaction is carried out by heating gradually to reflux a mixture of compound of formula 2, PEG-400 and an alkali metal carbonate in an organic solvent, cooled to ambient temperature and then ethyl chloroformate is added to it. The mixture is then heated for completion, to furnish the desired oxazolidinone (formula 1). The reaction mixture is worked up by standard methods known to those skilled in the art. The product is isolated with a yield of about 90%, and is greater than 99% pure 5-(3,5-dimethylphenoxy)methyl-2-oxazolidinone.

### Optional Further Purification of 5-(3,5-dimethylphenoxy)methyl-2-oxazolidinone

In another embodiment of the process of the present invention 5-(3,5-dimethylphenoxy)methyl-2-oxazolidinone (formula 1) is purified to greater than 99% purity to yield substantially pure 5-(3,5-dimethylphenoxy)methyl-2-oxazolidinone (formula 1) by recrystallization from a solvent, optionally by addition of a second solvent.

The solvent system which may be used in the purification step may comprise a mixture of solvents selected from a polar and non-polar organic solvent comprising of aliphatic, cyclic or aromatic substituted or unsubstituted hydrocarbons such as benzene, toluene, xylene, cyclohexane, dichloromethane, dichloroethane, monochlorobenzene and the like; alcohols such as C₁-C₆ alcohols like methanol, ethanol, propanols, butanols and the like; diols, polyols selected from ethylene glycol, propylene glycol and the like; esters such as ethyl acetate, butyl acetate and the like; ketones such as acetone, methlyisobutylketone, methylethylketone, cyclohexanone and the like; cyclic and acyclic ethers such as ether, tetrahydrofuran, dioxan, dimethoxyethane, diglyme and the like; polyethers such as poly(alkylene glycol) and the like; nitriles such as acetonitrile, benzonitrile and the like; amides such as dimethylformamide, dimethylacetamide and the like, sulfoxides such as dimethyl sulfoxide and the like. The preferred solvent system mixture for purification to achieve substantially pure 5-(3,5-dimethylphenoxy)methyl-2-oxazolidinone is a mixture comprising acetone and toluene, in the ratio ranging from 0.5:1.0 to 1:10, most preferably in the ratio 1:1.

Preferably, for recrystallization, the dissolution is carried out at about ambient to 110° C., more preferably about 50 to 80° C.

Optionally, to the clear solution may be added another solvent and cooled gradually or spontaneously to about 0 to 30° C., preferably to 15 to 25° C.

The crystallized product is filtered, washed with a solvent and dried using conventional techniques known to those skilled in the art to yield substantially pure 5-(3,5-dimethylphenoxy)methyl-2-oxazolidinone with purity greater than 99.9% by HPLC.

The substantially pure 5-(3,5-dimethylphenoxy)methyl-2-oxazolidinone has purity greater than 99.5% and has no individual impurity that is more than 0.05% by HPLC.

In the preferred process of the present invention crystallization is allowed to occur by chilling or seeding or scratching the glass of the reaction vessel or cooling and other such common techniques, preferably cooling.

The product may be dried using different techniques of drying like fluid bed drying, tray drying and rotatory drying techniques with or without application of vacuum and/or under inert conditions.

### Preparation of 3-(3,5-dimethylphenoxy)-2-hydroxypropylamine

### Step (a)—Preparation of 3-[(3,5-dimethylphenoxy)methyl]oxirane

According to the process of the present invention step (a) is carried out by reacting 3,5-dimethylphenol with epichlorohydrin and a base in a solvent, optionally in the presence of a facilitator.

The facilitator may be selected from quaternary ammonium salts such as benzyltrimethylammonium chloride and the like, or from cyclic and acyclic polyethers. Cyclic ethers such as crown ethers and acyclic ethers such as poly(alkylene glycol) may be used. Poly(alkylene glycol) which may be used is poly(ethylene glycol) (PEG) with an average molecular weight in the range between 200 to 10,000, preferably 200 to 1000, the most preferred being 400.

The solvent for the reaction could be an aliphatic, cyclic or aromatic substituted or unsubstituted hydrocarbons such as benzene, toluene, xylene, cyclohexane, dichloromethane, dichloroethane, monochlorobenzene and the like. In preferred embodiment, the solvent is a polar solvent comprising of cyclic and acyclic ethers such as ether, tetrahydrofuran, dioxan, dimethoxyethane, diglyme and the like; polyethers such as poly(alkylene glycol)s (PEG) such as PEG-200, PEG-400 and the like; nitriles such as acetonitrile, benzonitrile and the like; amides such as dimethylformamide, dimethylacetamide and the like, sulfoxides such as dimethyl sulfoxide and the like. In the present invention the preferred solvent is a water soluble ether, most preferably PEG-400, wherein no additional facilitator is required.

The base used could be selected from an organic or inorganic base, preferably an inorganic base selected from bicarbonates, carbonates, hydrides, hydroxides and oxides of alkali or alkaline earth metals. Most preferably the base is potassium hydroxide.

Further, the reaction may be carried out at about 20 to 80° C. The preferred temperature of step (a) may be 25 to 60° C., the most preferred being 35 to 45° C.

The reaction may be carried out in poly(ethylene glycol)-400 in the presence of a base.

The reaction may be carried out in poly(ethylene glycol)-400 in the presence of potassium hydroxide at 35 to 45° C.

The reaction mixture is worked up by standard methods known to those skilled in the art.

### Step (b)—Preparation of 3-(3,5-dimethylphenoxy)-2-hydroxypropylamine

According to the process of the present invention step (b) is carried out by reacting 2-[(3,5-dimethylphenoxy)methyl]oxirane with ammonia, preferably in a solvent. Ammonia could be used in the form of liquor ammonia, liquid ammonia or ammonia gas.

According to one embodiment of the present invention the organic solvent is selected from polar solvents like; alcohols such as C₁-C₆ alcohols like methanol, ethanol, propanols, butanols and the like; diols, polyols selected from ethylene glycol, propylene glycol and the like; ketones such as acetone, methlyisobutylketone, methylethylketone, cyclohexanone and the like; cyclic and acyclic ethers such as ether, tetrahydrofuran, dioxan, dimethoxyethane, diglyme and the like; polyethers such as poly(alkylene glycol)s and the like; nitriles such as acetonitrile, benzonitrile and the like; amides such as dimethylformamide, dimethylacetamide and the like, sulfoxides such as dimethyl sulfoxide and the like. When liquor ammonia is used polar water soluble solvents are preferred.

In a preferred embodiment of the present invention organic solvent is an alkanol selected from C₁ to C₄ alkanol or its admixture with water. More preferably the alkanol is methanol.

Preferably, step (a) is carried out by adding a solution of 2-[(3,5-dimethylphenoxy)methyl]oxirane in methanol to a stirred solution containing large molar excess of liquor ammonia and methanol slowly over a period of about 9 hours while maintaining the temperature of about 25 to 30° C.

The reaction mixture is worked up by standard methods known to those skilled in this art. For instance, in a specific embodiment after completion of reaction methanol was distilled out below 60° C. under vacuum. The product was extracted into methylene dichloride and the organic extract was acidified with conc. HCl added till about pH 2 to precipitate the product selectively as a hydrochloride salt devoid of impurities, which could be easily filtered to get 99% pure 3-(3,5-dimethylphenoxy)-2-hydroxypropylamine hydrochloride.

The invention is illustrated but not restricted by the description in the following example.

### EXAMPLES

### Example-1

### (a) Preparation of 2-[(3,5-Dimethylphenoxy)methyl]oxirane (Formula 5)

To a stirred solution of 3,5-dimethylphenol (10 g, 0.818 mol.), PEG-400 (300 ml), epichlorohydrin (128.01 ml, 1.63 mol) at 25-30° C. is added one part of potassium hydroxide (18.37 g, 0.32 mol.). Two more lots of potassium hydroxide (18.37 g each, 0.64 mol.) are charged, each after an hour's interval after cooling the mixture to 25-30° C. The mixture was then stirred further for an hour of at 35-45° C. Water (400 ml) is slowly added and the product is extracted into hexane (2×200 ml) and (1×100 ml). The combined hexane extract is concentrated at 60-65° C. under vacuum. Any excess epichlorohydrin in the residue is finally stripped off by adding toluene (50.0 ml) and degassing at 60-65° C. under vacuum. Yield of the product is 142.0 g.

### (b) Preparation of 3-(3,5-Dimethylphenoxy)-2-hydroxypropylamine hydrochloride (Formula 2)

A solution of 2-[(3,5-dimethylphenoxy)methyl]oxirane (100.0 g, 0.561 mol) in methanol (300.0 ml) is added slowly during about 9 hrs, to a stirred solution containing liquor ammonia (1150 ml) and methanol (700 ml) while maintaining the temperature between 25-30° C. After completion of addition, the mixture is stirred for a further 1 hr and the methanol was distilled out under reduced pressure at below 60° C. The product is then extracted into methylene dichloride (1×300 ml. & 1×200 ml.). Pooled extracts are washed with water (2×250 ml). The organic layer is dried over anhydrous sodium sulfate, cooled to 5-10° C. and conc. HCl is added until the pH is about 2.0. The precipitated hydrochloride salt is filtered, washed successively with methylene dichloride (100.0 ml) and hexane (50.0 ml). Product is finally dried in air oven at 75-80° C. to yield 3-(3,5-dimethylphenoxy)-2-hydroxypropylamine hydrochloride, 62.0 g (purity>99.0%)

### (c) Preparation of 5-(3,5-dimethylphenoxy)methyl-2-oxazolidinone (Formula 1)

A mixture of PEG400 (50 ml), toluene (500 ml), potassium carbonate powder (89.6 g, 0.648 mol), and 3-(3,5-dimethylphenoxy)-2-hydroxypropylamine hydrochloride (formula 3) (50 g, 0.216 mol) is heated gradually to reflux during 1.0 hr., and then azeotropically refluxed for 3 hrs. The mixture is then cooled to 25-30° C. and ethyl chloroformate (formula 4) (24.8 g, 0.228 mol.) is added gradually during 6 hrs. while maintaining the temperature below 40° C. during the addition. The reaction mixture is then heated at 50-55° C. for 2 hours. The temperature is raised to reflux and then refluxed azeotropically for 5.0 hrs using Dean-Stark condenser. The mixture is then cooled to 10-15° C., water (150 ml) is added and the pH is adjusted to 6.5-7.0 by gradual addition of conc. HCl. After stirring at 10-15° C. for 1 hr. the product is separated by filtration and washed with toluene (2×25 ml), followed by water until washings are free from chloride, and dried. The toluene layer from the filtrates is separated, washed with water (2×100 ml). It is concentrated to one tenth of the volume, cooled to 25-30° C. and the crystallized second crop is filtered. Yield of product 43.0 g (90%, purity>99% by HPLC).

### (d) Purification of 5-(3,5-dimethylphenoxy)methyl-2-oxazolidinone (Formula 1)

5-(3,5-dimethylphenoxy)methyl-2-oxazolidinone (formula 1), (5 g) obtained in example 1(c) is dissolved in acetone (15 ml) by heating to 60-65° C. To the clear solution is added toluene (15 ml), cooled gradually to 20-25° C. and stirred for 2 hrs. at this temperature. The crystallized product is filtered, washed with toluene (5 ml) and dried to get 5-(3,5-dimethylphenoxy)methyl-2-oxazolidinone (formula 1) with purity of 99.93% (having a maximum single impurity of 0.02%).

### Example 2

### Method for Analysis of Purity by HPLC Buffer

Dissolve 1.36 g potassium dihydrogenorthophosphate in 1000 ml of water. Take 650 ml of buffer add 2 ml of triethylamine. Adjust pH to 2.5 by orthophosphoric acid.

Mobile Phase:

Mix buffer solution and acetonitrile in the ratio of 650:350. Filter and degas prior to use.

Sample Preparation:

Transfer about 100 mg accurately weighed sample into a 100 ml volumetric flask. Dissolve in and dilute upto mark with mobile phase.

System Suitability Solution:

Transfer about 10 mg of metaxalone into a 100 ml volumetric flask. Dissolve in and dilute upto mark with mobile phase.

Chromatographic System:

The liquid chromatograph is equipped with a 225 nm UV detector and 25 cm×4.6 mm, 5 micron column that contains Hypersil BDS C8. The flow rate is about 1.0 ml/min.

Procedure:

Inject 10 ml of system suitability solution into the system and record the chromatograms upto 25 min. Calculate the tailing factor of metaxalone peak. It should not be more than 1.5 and number of theoretical plates should not be less than 5000.

Inject 10 ml of sample preparation into the system and record the chromatograms upto 25 min. The retention time of metaxalone is 13 min. Calculate the amount of related substances by area normalization method, while disregarding any peak with an area percentage less than 0.025.

1. (Cancelled)

2. (Cancelled)

3. (Cancelled)

4. (Cancelled)

5. (Cancelled)

6. (Cancelled)

7. (Cancelled)

8. (Cancelled)

9. (Cancelled)

10. (Cancelled)

11. (Cancelled)

12. (Cancelled)

13. (Cancelled)

14. (Cancelled)

15. (Cancelled)

16. (Cancelled)

17. (Cancelled)

18. (Cancelled)

19. (Cancelled)

20. (Cancelled)

21. (Cancelled)

22. (Cancelled)

23. (Cancelled)

24. (Cancelled)

25. (Cancelled)

26. (Cancelled)

27. (Cancelled)

28. A novel process for the preparation of 5-(3,5-dimethylphenoxy)methyl-2-oxazolidinone (formula 1) comprising 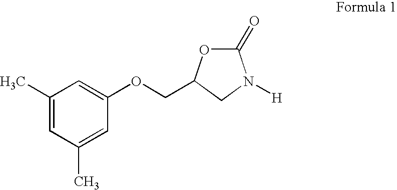
reacting 3-(3,5-dimethylphenoxy)-2-hydroxypropylamine, compound of formula 2, or its acid addition salt with a compound of formula 3, 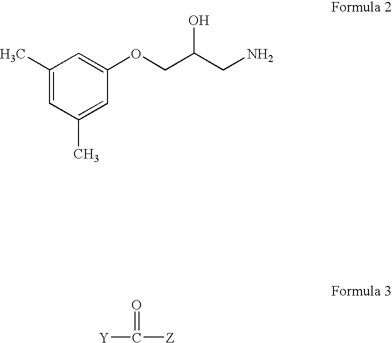
wherein Y and Z are selected from X, CCl ₃CO, 1-imidazolyl or substituted imidazolyl, and OR: wherein X is a halo radical, and R is selected from a substituted or unsubstituted linear, branched or cyclic alkyl radical, and aryl or heteroaryl radical.

29. A process as claimed in claim 28 wherein the reaction is carried out in the presence of a base.

30. A process as claimed in claim 29 wherein the base is potassium carbonate . . .

31. A process as claimed in claim 28 wherein in the compound of formula 3 Y is a halo radical and Z is OR wherein is a linear C₁ to C₄ alkyl radical.

32. A process claimed in claim 31 wherein the compound of formula 3 is ethyl chloroformate.

33. A process as claimed in claim 28 wherein the reaction is carried out in the presence of a facilitator.

34. A process as claimed in claim 33 wherein the facilitator is selected from cyclic and acyclic polyethers.

35. A process as claimed in claim 34 wherein the facilitator is poly(ethylene glycol) with an average molecular weight in the range between 200 to 10,000.

36. A process as claimed in claim 28 wherein the molar ratio of compound of formula 2 to compound of formula 3 is in the range of about 1:0.8 to 1:1.5.

37. A process as claimed in claim 28 wherein the 5-(3,5-dimethylphenoxy)methyl 2-oxazolidinone (formula 1) is obtained in a substantially pure form and has a purity greater than 99%.

38. A process for purifying 5-(3,5-dimethylphenoxy)methyl-2-oxazolidinone (formula 1) by crystallizing 5-(3,5-dimethylphenoxy)methyl-2-oxazolidinone (formula 1) from an organic solvent system.

39. A process as claimed in claim 38 wherein the 5-(3,5-dimethylphenoxy)methyl-2-oxazolidinone (formula 1) is obtained in a substantially pure form and has a purity greater than 99.5%.

40. A process as claimed in claim 38 wherein the 5-(3,5-dimethylphenoxy)methyl-2-oxazolidinone (formula 1) is obtained in a substantially pure form has a purity greater than 99.9%.

41. A process as claimed in claim 38 wherein the 5-(3,5-dimethylphenoxy)methyl-2-oxazolidinone (formula 1) is obtained in a substantially pure form and his a purity greater than 99.5% and no individual impurity that is more t 0.05%.

42. A process as claimed in claim 38 wherein the organic solvent system is a mixture of acetone and toluene.

43. A process as claimed in claim 42 wherein the volume ratio of acetone toluene is about 0.5:1.0 to 1:10.
